(19) United States
(12) Patent Application Publication
(10) Pub. No.: US 2011/0130768 A1
(43) Pub. Date: Jun. 2, 2011

(54) IDENTIFICATION TAG LOADING MEANS

(76) Inventors: John Glenny Douglas Ritchie, Waihi (NZ); Stephen Michael Veith, Currambine (AU)

(21) Appl. No.: 12/750,615

(22) Filed: Mar. 30, 2010

(30) Foreign Application Priority Data
Dec. 2, 2009 (AU) AU 20092433473

(51) Int. Cl. A61B 17/00

(52) U.S. Cl. 606/117

(57) ABSTRACT

An identification tag loading means (1) includes a chamber (5, 7) in which the head portion (12) and cutting tip (13) of a one piece animal tag (8) can be accommodated. At this time the aperture (11) at the outer end of the hollow shank (10) is exposed and ready for loading the tag (8) onto the applicator pin (14) of an ear tag applicator (15). This reduces the risk of damage to a users hands by contact with the tip (13).

### BACKGROUND OF THE INVENTION

The present invention relates to an identification tag loading means and more particularly, means for the loading of a one piece animal identification tag onto an applicator.

One piece tags are well known, such as that of New Zealand Patent 226296/228456 of Michael Stuart Gardner. Such tags feature a head with a piercing tip which forms a clean cut in the animal's ear, as the tip passes through the animal's ear for the head to be retained on the opposite side of the ear, while the flag portion of the tag incorporating the animal identification indicia remains on the opposite side of the ear.

Because the tip is required to surgically pierce the animal's ear, it requires careful handling by a user in order to protect his or her hands, and this inevitably results in the tagging operation being slowed down.

### OBJECTS OF THE INVENTION

It is thus an object of one embodiment of the present invention to provide an identification tag loading means, or method of loading, which enables the loading of a one piece tag onto an applicator and which avoids or at least obviates problems with present animal tag loading means or methods or which at least will provide the public with a useful choice.

### BRIEF SUMMARY OF THE INVENTION

According to one aspect of the present invention there is provided an identification tag loading means for loading a one piece tag onto an applicator, said loading means including a body portion including, at least at one end, an opening into a chamber, said chamber being adapted to accommodate, in use, a head portion of said tag such that an aperture of the tag is presented for the introduction of a pin of the applicator into the tag and wherein the chamber is further adapted to subsequently permit the release of the head portion from the chamber to leave the tag loaded on the pin.

Preferably, the chamber may include a first part into which, in use, at least part of a tip of the head portion may protrude and a second part, of larger dimensions than the first part, within which at least a substantial portion of the remainder of the head portion can be accommodated.

Preferably, the chamber may be substantially cylindrical.

Preferably, a first inner part of the chamber may have a first diameter and an outer second part may have a larger second diameter.

Preferably, an inwardly tapered wall portion connects the second and first parts of the chamber.

According to a second aspect of the present invention there is provided a method of loading a one piece identification tag on an applicator, said method including:
i. Providing a loading means with a chamber adapted to removably accommodate, in use, a head portion of the tag;
ii. Positioning the head portion within the chamber so that an aperture of the tag, leading to a substantially hollow shank portion, is externally presented;
iii. Inserting a pin of the applicator into the aperture;
iv. Rotating the loading means so that the head portion is substantially aligned with the shank portion;
v. Causing the movement of the pin relative to and through the shank portion to mount the head portion on the pin; and
vi. Removing the loading means from the tag to leave the tag loaded on the pin.

According to a still further aspect of the present invention there is provided an identification tag loading means and/or a method of loading an identification tag on an applicator, substantially as herein described and with reference to the accompanying drawings.

Further aspects of this invention, which should be considered in all its novel aspects, will become apparent from the following description, given by way of example of possible embodiments of the invention, and in which reference is made to the accompanying drawings.

### BRIEF DESCRIPTION OF THE DRAWINGS

FIGS. 1(a)-(d): show respectively, perspective, side, cross sectional and end views of a loading means according to one possible embodiment of the invention;

FIG. 2: shows diagrammatically, and in part cross-section, a one piece tag having been loaded into the loading means of FIG. 1;

FIG. 3: shows the one piece tag of FIG. 2 now introduced onto the end of a pin of an applicator;

FIG. 4: shows the loading means of FIG. 3 now rotated relative to the applicator pin;

FIG. 5: shows the loading means of FIG. 4 having pushed the one piece tag into position on the applicator pin;

FIG. 6: shows the loading means of FIG. 5 now withdrawn from the one piece tag; and

FIG. 7: shows the loaded applicator now ready to tag an animal's ear.

### BRIEF DESCRIPTION OF POSSIBLE EMBODIMENTS

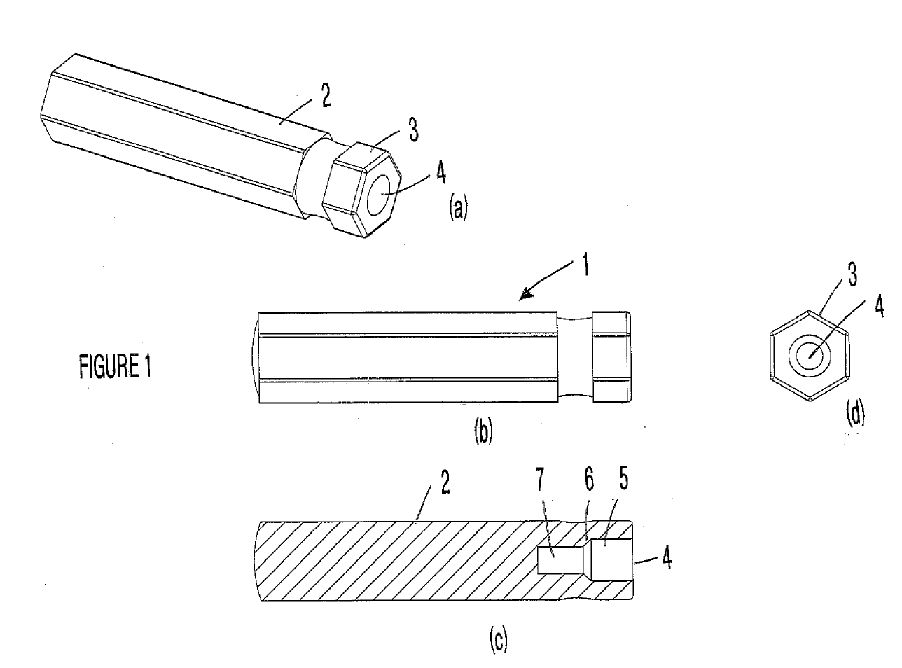

Referring firstly to FIG. 1 a loading means, according to one possible embodiment of the invention, is referenced generally by arrow 1. The loading means 1 may be manufactured of any suitable material and by any suitable means but particularly suitable is a moulding of a hard plastics. The loading means 1 is shown with an elongate body portion 2 having an end portion 3 which may, as shown, be of a hexagonal shape, which may facilitate gripping by a user.

The end portion 3 is shown with an opening 4 into a chamber which may, as shown, include a first outer chamber 5, leading to an inwardly tapered wall portion 6 and an inner chamber 7 of a smaller size. As shown, both the inner and outer chambers 7, 5 are substantially cylindrical, the inner chamber 7 being of a smaller diameter.

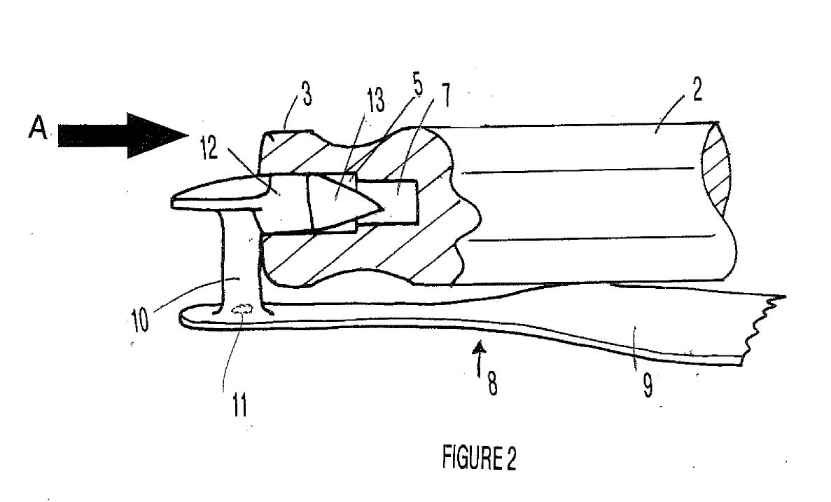

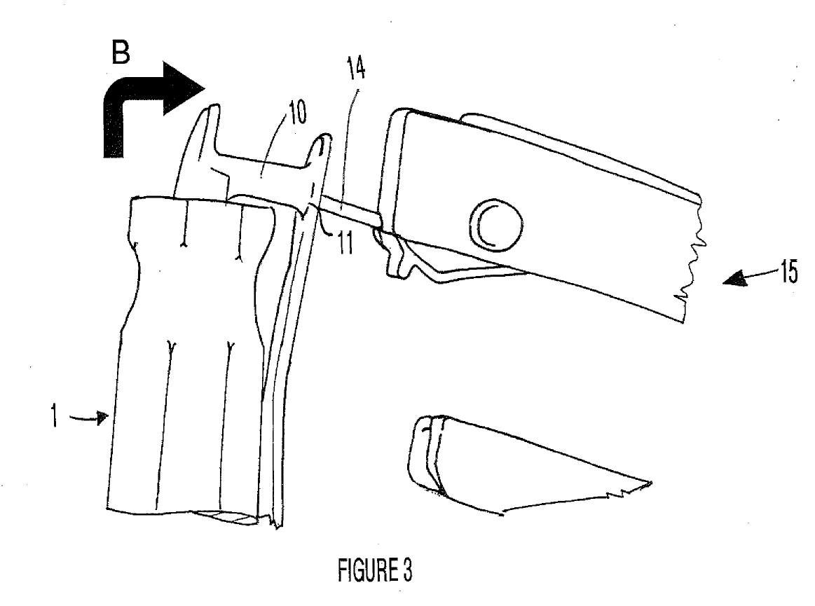

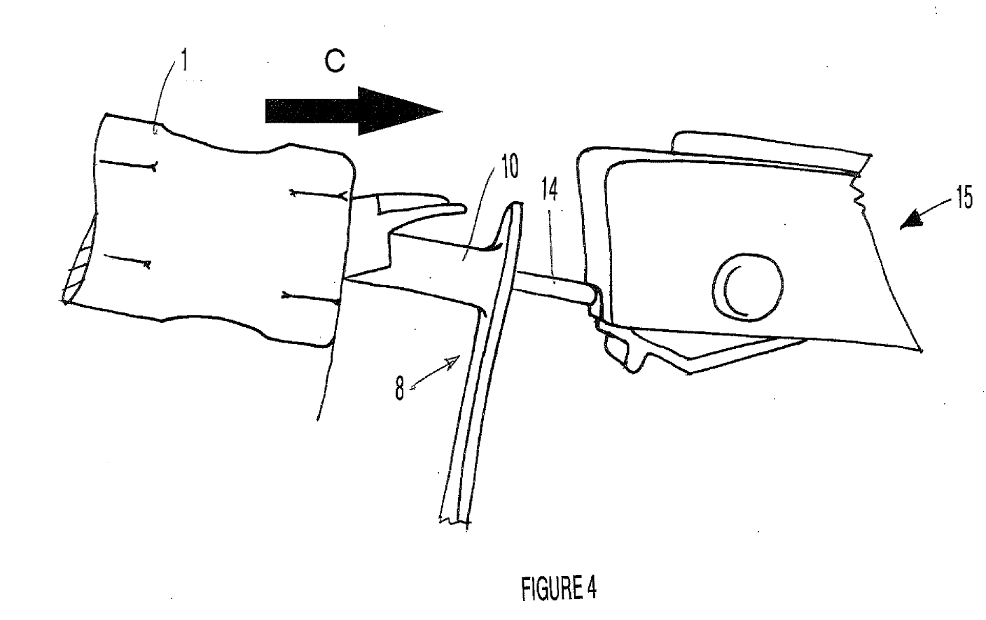

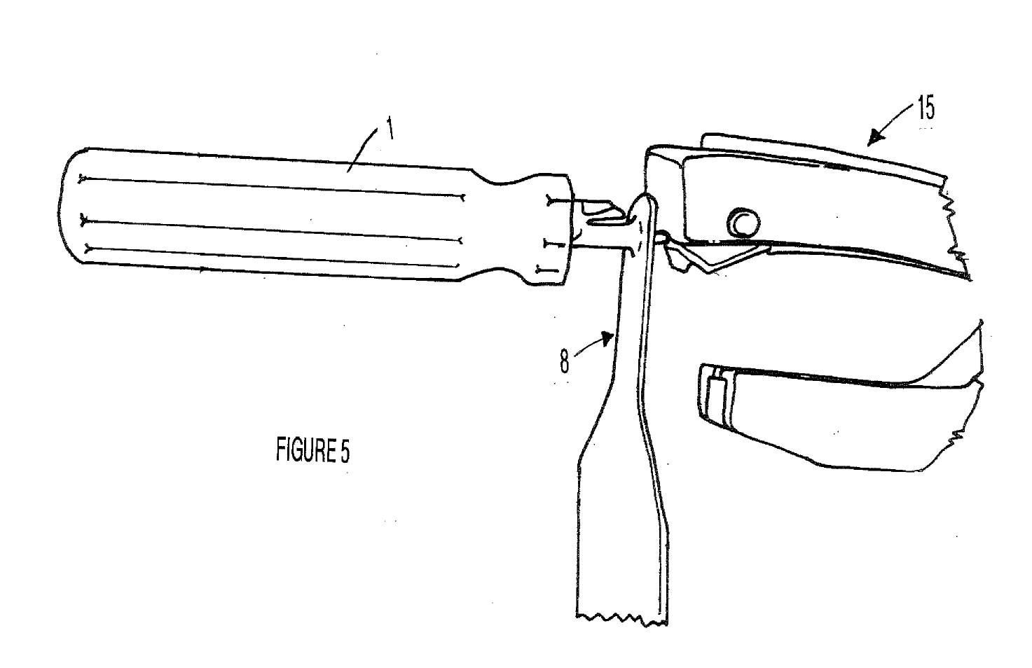

Turning then to FIG. 2, a user, after gripping the loading means 1, has inserted the head portion 12 of a one piece tag 8 into the chamber 5 so that the end of its cutting tip portion 13 is extending into the inner chamber 7. In this position the substantially hollow shank 10, extending from the head portion 12, is positioned adjacent the outer surface of the end portion 3, with the flag portion 9 of the one piece tag 8 lying adjacent the body portion 2. In this position the aperture 11 at the end of the hollow shank 10 is in an exposed position for the reason which will now become clear. In particular, referring to FIG. 3, the pin 14 of a tag applicator 15 is shown inserted into the opening 11 at which time the loading means 1 can be rotated in the direction indicated by arrow B and into the position shown in FIG. 4. In this latter position the loading means 1 can be pushed in the direction indicated by arrow C so that the shank portion 10 moves along and is firmly located over the applicator pin 14 and as shown in FIG. 5.

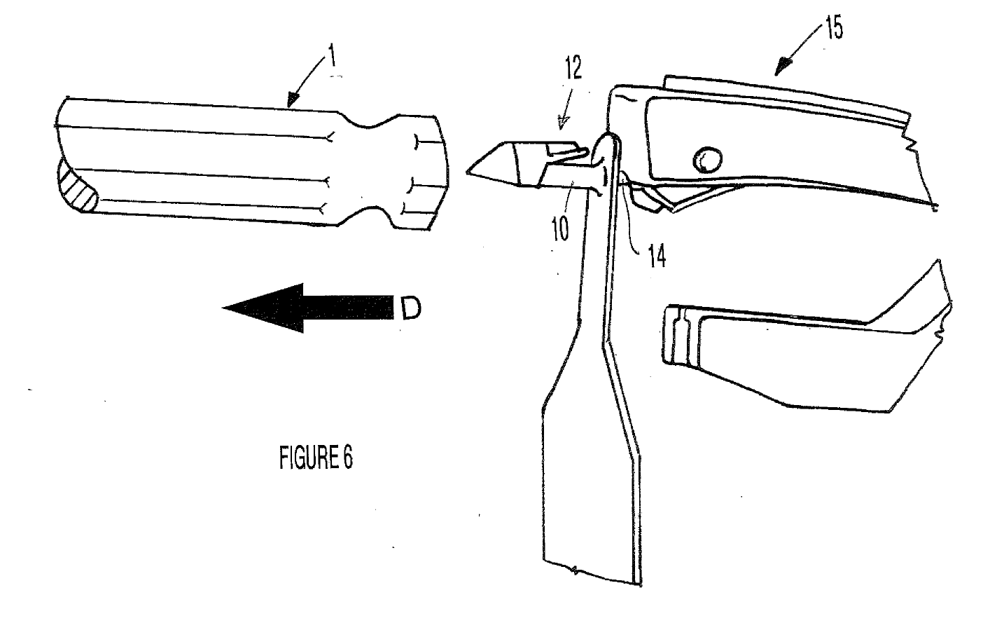

In this position, the loading means 1 can be withdrawn, as shown in FIG. 6, in the direction indicated by arrow D, leaving the head portion 12 and the shank portion 10 firmly mounted on the applicator pin 14.

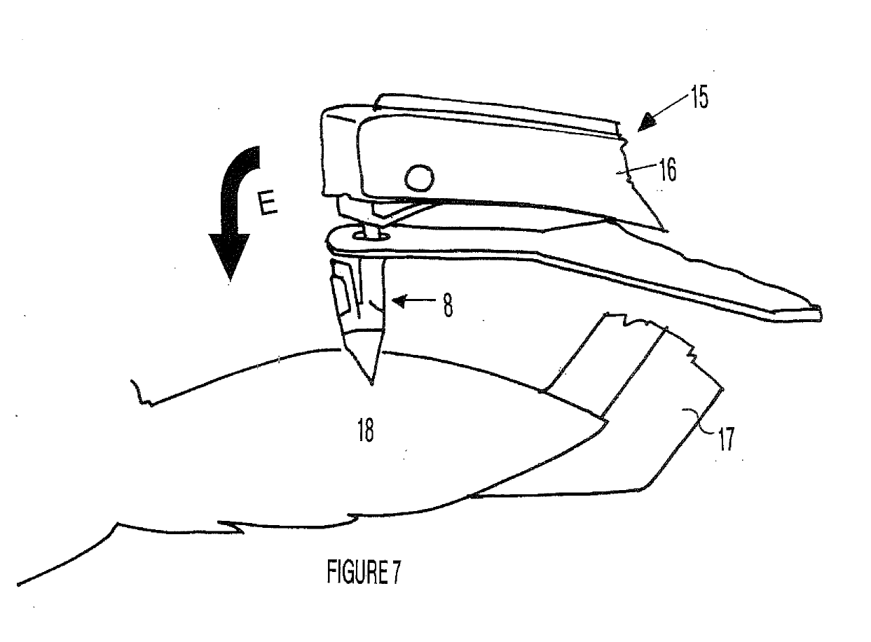

From this position, the applicator pin 14 can be pivoted downwardly in a direction indicated by arrow E, into the position shown in FIG. 7, and ready for the tagging of the tag 8 to the animal's ear 18 by the closing of the jaws 16 and 17 of the applicator 15.

It is seen that apart from the act of locating the head portion 12 within the loading means 1, a user has been able to avoid having to risk damaging their hands by inadvertent contact with the piercing tip 13. Moreover, that once the head portion 12 of the one piece tag 8 has been inserted into the loader 1, the loading of the applicator can be performed simply yet effectively.

Where in the foregoing description, reference has been made to specific components or integers of the invention having known equivalents, then such equivalents are herein incorporated as if individually set forth.

Although this invention has been described by way of example and with reference to possible embodiments thereof, it is to be understood that modifications or improvements may be made thereto without departing from the scope of the invention, as defined in the appended claims.

1. An identification tag loading means for loading a one piece tag onto an applicator, said loading means including a body portion including, at least at one end, an opening into a chamber, said chamber being adapted to accommodate, in use, a head portion of said tag such that an aperture of the tag is presented for the introduction of a pin of the applicator into the tag and wherein the chamber is further adapted to subsequently permit the release of the head portion from the chamber to leave the tag loaded on the pin.

2. A loading means as claimed in claim 1 wherein the chamber includes a first part into which, in use, at least part of a tip of the head portion can protrude and a second part, of larger dimensions than the first part, within which at least a substantial portion of the remainder of the head portion can be accommodated.

3. A loading means as claimed in claim 2 wherein the chamber is substantially cylindrical.

4. A loading means as claimed in claim 3 wherein a first inner part of the chamber has a first diameter and a second outer part of the chamber has a larger second diameter.

5. A loading means as claimed in claim 4 wherein an inwardly tapered wall portion connects the second and first parts of the chamber.

6. A method of loading a one piece identification tag on an applicator, said method including:
i. Providing a loading means with a chamber adapted to removably accommodate, in use, a head portion of the tag;
ii. Positioning the head portion within the chamber so that an aperture of the tag, leading to a substantially hollow shank portion, is externally presented;
iii. Inserting a pin of the applicator into the aperture;
iv. Rotating the loading means so that the head portion is substantially aligned with the shank portion;
v. Causing the movement of the pin relative to and through the shank portion to mount the head portion on the pin; and
vi. Removing the loading means from the tag to leave the tag loaded on the pin.
